(19) United States
(12) Patent Application Publication
(10) Pub. No.: US 2010/0086437 A1
(43) Pub. Date: Apr. 8, 2010

(54) PROCESSES FOR DEACTIVATING MICROORGANISMS IN SOLID MATERIALS

(75) Inventors: Abderrahmane DERMOUNE, Sherbrooke (CA); Soraya MAHIOUT, Sherbrooke (CA); Roger PARADIS, Mont St-Hilaire (CA)

Correspondence Address: Anglehart et al., Suite 258, 393 St-Jacques, Montreal, QC, H2Y 1N9, CA

(73) Assignee: LES TECHNOLOGIES ELCOTECH INC., Boucherville, QC (CA)

(21) Appl. No.: 12/356,762

(22) Filed: Jan. 21, 2009

Related U.S. Application Data
(63) Continuation of application No. PCT/CA2007/001302, filed on Jul. 20, 2007.
Application No. PCT/CA2007/001302 is a continuation-in-part of application No. 10/902,877, filed on Aug. 2, 2004.
Application No. 10/902,877 is a continuation-in-part of application No. 10/915,491, filed on Aug. 11, 2004, now Pat. No. 7,578,918.
(60) Provisional application No. 60/820,009, filed on Jul. 21, 2006.

(51) Int. Cl. A61L 2/03

(52) U.S. Cl. 422/22

(57) ABSTRACT

There is provided a process for at least partially deactivating microorganisms in a solid material. The process comprising submitting the solid material to an electric current having a voltage gradient of at least 3 volts per centimeter of solid material to be treated, and a current density of at least 2 mA per cm² with respect to the surface of electrodes used for generating the current.

### TECHNICAL FIELD

The present invention relates to improvements in the field of solid materials decontamination treatment. In particular, the invention relates to processes for microorganisms deactivation and/or destruction in solid materials. Such processes can be useful for treating various types of solid materials, such as sludges. They also permit deactivation and/or destruction of various types of pathogenic microorganisms.

### BACKGROUND OF THE INVENTION

The management and reclamation of municipal sludge generated by wastewater treatment require a beforehand dewatering and pathogens reduction.

The principal microorganisms representative and indicators of treatment efficiency for pathogens reduction are: fecal coliforms or Salmonella sp., enteric viruses and viable helminth ova (US EPA, 2003. Control of Pathogens and Vector Attraction in Sewage Sludge. Environmental Regulations and technology. United States Environmental Protection Agency. EPA/625/R-92/013. Revised July 2003). All those microorganisms are likely to be present at various degrees in municipal sludge.

According to US EPA (1993), the aim of class A processes is to reduce pathogen densities below the following detection limits: less than 3 most probable number (MPN) per 4 grams of total solid (dry weight basis) for Salmonella sp.; less than 1 plaque-forming unit (PFU) per 4 grams of total solids (dry weight basis) for enteric viruses and less than 1 viable helminth ova per 4 grams of total solid (dry weight basis). For fecal coliforms, used as indicator organism, the aim is to reduce them to less than 1000 most probable number (MPN) per 1 gram of total solid (dry weight basis).

Other microorganisms like Clostridium perfringens, aerobic and facultative anaerobic heterotrophic bacteria, total coliforms, enterococus and coliphages can be used as indicator organisms. Coliphages (bacteriophages) are viruses that infect bacteria. Thanks to their characteristics, coliphages are considered by researchers as indicators of fecal pollution and are proposed as a model to follow the fate of enteric viruses in various hydrous environment and wastewater treatment plant.

Actual technologies for sludge sanitization rely essentially on thermal treatment, heat drying and heat treatment, high pH combined to high temperature process (alkaline treatment), thermophilic digestion, beta and gamma ray irradiation, pasteurization and composting.

It is thus highly desirable to be provided with an alternative to the solutions proposed so far.

### SUMMARY OF THE INVENTION

According to one aspect of the invention, there is provided a process for at least partially deactivating microorganisms in a solid material. The process comprises submitting the solid material to an electric current having a voltage gradient of at least 3 volts per centimeter of solid material to be treated, and a current density of at least 2 mA per cm² with respect to the surface of electrodes used for generating the current.

It was found that such a process is very useful to treat solid materials in order to at least partially deactivate the microorganisms contained therein or even destroy the microorganisms contained therein. The process represents an economic and simple manner to decontaminate or to permit hygienization of solid materials. The process can permit to deactivate and/or destroy a plurality of microorganisms such as fecal coliforms, Salmonella spp., enteric viruses, viable helminth ova, enterococus, aerobic and facultative anaerobic heterotrophic bacteria, total coliforms, Escherichia coli, Clostridium perfringens, somatic coliphages and F-specific coliphages. The efficiency of the process is quite impressive since it can permit to considerably decrease the amount of microorganisms present in the solid material. The costs and problems related to the disposal of a solid material contaminated with microorganisms can thus be avoided by using such a process.

According to another aspect of the invention, there is provided a process for at least partially deactivating microorganisms in a solid material, and dewatering the solid material. The process comprises submitting the solid material to an electric current having a voltage gradient of at least 3 volts per centimeter of solid material to be treated, and a current density of at least 2 mA per cm² with respect to the surface of electrodes used for generating the current density and compacting the solid material.

It was found that such a process is very useful to treat solid materials in order to dewater it and to at least partially reduce the amount of microorganisms contained therein or even destroy the microorganisms contained therein. The process represents an economic and simple manner to dewater and decontaminate or to permit hygienization of solid materials. The process can permit to reduce or eliminate a plurality of microorganisms such as fecal coliforms, Salmonella spp., enteric viruses, viable helminth ova, enterococus, aerobic and facultative anaerobic heterotrophic bacteria, total coliforms, Escherichia coli, Clostridium perfringens, somatic coliphages and F-specific coliphages. The process can permit to at least partially eliminate or decrease the amount of microorganisms present in the solid material. Thus, such a process is very useful since it can convert a contaminated solid material having a considerable volume of water or solution i.e. low dryness into a dewatered solid material with higher dryness, reduced volume and in which the amount of microorganisms has been considerably reduced or in which the microorganisms have been eliminated. Therefore, the costs and difficulties to dispose and handle a solid material are considerably lowered when the solid material is treated with such a process.

The expression “deactivating microorganisms” as used herein refers to preventing the microorganisms from being active and causing undesired effects. For example, “deactivating microorganisms” can refer to preventing the microorganisms from metabolizing and/or multiply. Deactivation of microorganisms can also include reduction, elimination or destruction of the microorganisms.

The voltage gradient can be at least 4 V/cm, at least 5 V/cm, at least 6 V/cm, at least 7 V/cm, at least 8 V/cm, at least 9 V/cm, at least 10 V/cm, at least 12 V/cm, at least 14 V/cm, at least 16 V/cm, at least 18 V/cm at least 20 V/cm, at least 22 V/cm, at least 24 V/cm, at least 26 V/cm, at least 28 V/cm, or at least 30 V/cm. Alternatively, the voltage gradient can be about 3 V/cm to about 50 V/cm, 3 V/cm to about 60 V/cm, 3 V/cm to about 80 V/cm or about 15 V/cm to about 30 V/cm. The current can have an average current density of at least 3 mA/cm², at least 4 mA/cm², at least 5 mA/cm² at least 6 mA/cm², at least 7 mA/cm², at least 8 mA/cm², at least 9 mA/cm², at least 10 mA/cm², at least 12 mA/cm², at least 14 mA/cm², at least 16 mA/cm², at least 18 mA/cm², at least 20 mA/cm², at least 25 mA/cm² or at least 30 mA/cm². Alternatively, the average current density can be of about 6 mA/cm² to about 70 mA/cm² or of about 10 mA/cm² to about 45 mA/cm².

The current can be applied to the solid material for a period of at least 5 minutes, at least 12 minutes, at least 20 minutes, or at least 25 minutes. The solid material can be submitted to the electrical current and compacted simultaneously. The solid material can be compacted by a pressure applied to it, the pressure varying according to the consistency of the solid material, the pressure increasing when the solid material consistency is increasing. The pressure applied to the solid material can be substantially non-existent at the beginning of the process, and then, the pressure is progressively increased. A pressure of at least about 0.1 bar can be applied to the solid material in order to compact it while submitting it to the electric current. The pressure can also be about 0.15 bar to about 5 bars. Alternatively, the pressure applied to the solid material can be constant.

The solid material can be compressed by maintaining a contact substantially constant between at least one electrode and the solid material when the solid material is submitted to the electric current.

The solid material, before the treatment, can have a dryness of at least 25%. The dryness can also be about 2% to about 25%. For example, the solid material, after the treatment, can have a dryness increased of at least 10% as compared to the dryness of the solid material before the treatment.

The processes of the present invention can further comprise imparting a rotation movement to the solid material while the material is being compacted and submitted to the electrical current.

Alternatively, the solid material can be moved in a predetermined direction, and the solid material can be compacted by applying a pressure to the solid material in a direction which is substantially perpendicular to the predetermined direction.

The processes can permit to reduce of at least 70%, at least 99%, at least 99.9%, at least 99.99%, at least 99.999%, or at least 99.9996% the content of Escherichia coli in the solid material. The processes can permit to reduce of at least 70%, at least 99%, at least 99.9%, at least 99.99%, at least 99.999%, or at least 99.9999 the content of Salmonella spp. in the solid material. The processes can permit to reduce of at least 70%, at least 99%, at least 99.9%, or at least 99.99% the content of enterococus in the solid material. The processes can permit to reduce of at least 70%, at least 97%, at least 99%, or at least 99.99% the content of aerobic and facultative anaerobic heterotrophic bacteria in the solid material. The processes can permit to reduce of at least 70%, at least 99%, at least 99.9%, at least 99.99%, at least 99.999%, or at least 99.9999% the content of total coliforms in the solid material. The processes can permit to reduce of at least 70%, at least 99%, at least 99.9%, at least 99.99%, at least 99.999%, or at least 99.9999% the content of fecal coliforms in the solid material. The processes can permit to reduce of at least 70%, at least 99%, at least 99.9%, at least 99.99%, or at least 99.999% the content of Clostrodium perfringens in the solid material. The processes can permit to reduce of at least 70%, at least 99%, or at least 99.9% the content of somatic coliphages in the solid material. The processes can permit to reduce of at least 70%, at least 99%, at least 99.9%, or at least 99.99% the content of F-specific coliphages in the solid material. The processes can permit to reduce of at least 70%, at least 94%, at least 99.9%, at least 99.99%, or at least 99.9999% the content of enteric viruses in the solid material. The processes can permit to reduce of at least 70%, at least 99%, at least 99.9%, at least 99.99%, at least 99.999% or at least 99.9999% the content of helminths ova in the solid material. The processes can also permit to eliminate helminths ova in the solid material.

The processes can permit to substantially prevent the microorganisms from metabolizing and/or multiply and/or to substantially reduce or eliminate the presence of microorganisms in the solid material. Moreover, the processes can permit to substantially destroy microorganisms in the solid material.

One way to meet the Class A pathogen reduction requirements is to treat sewage sludge in a process equivalent to the processes to further reduce pathogens (US EPA, 2003. Control of Pathogens and Vector Attraction in Sewage Sludge. Environmental Regulations and technology. United States Environmental Protection Agency. EPA/625/R-92/013. Revised July 2003). One of the processes to further reduce pathogens (PFRP) is pasteurization that involves heating sewage sludge to above a predetermined temperature for a minimum time period. During pasteurization, sludge temperature is maintained at 70° C. or higher for 30 minutes or longer.

During the treatment, the processes of the present invention can permit to submit the solid material to a temperature higher than 70° C. For example, the processes can be considered as equivalent to a process to further reduce pathogens (PFRP) if they comprise certain parameters. The two following examples represent such processes.

Firstly, the processes can comprise submitting the solid material to an electric current having a voltage gradient of at least 3 volts per centimeter of solid material to be treated, and a current density of at least 2 mA per cm² with respect to the surface of electrodes used for generating the current density, and compacting the solid material and submitting the solid material to a temperature of at least 70° C. for at least 30 minutes.

Secondly, the processes can comprise submitting the solid material to an electric current having a voltage gradient of at least 3 volts per centimeter of solid material to be treated, and a current density of at least 2 mA per cm² with respect to the surface of electrodes used for generating the current density, and compacting the solid material and submitting the solid material to a temperature of at least 70° C. for a time period t₁, wherein t₁≦30 minutes. A system, for maintaining the temperature of the solid material at 70° C. or more, can then be added at the end of the treatment. The solid material is thus subjected to a temperature of 70° C. or more during a time period t₂ (t₂ 30 minutes) in such a way that the time period t₃ (t₃=t₁+t₂) is at least 30 minutes. Alternatively, t₁<30 minutes; and t₂<30 minutes.

According to another aspect of the invention, there is provided a process for at least partially deactivating microorganisms in a solid material, the process comprising submitting the solid material to an electric current having a voltage gradient of at least 0.5 volt per centimeter of solid material to be treated, and a current density of at least 2 mA per cm² with respect to the surface of electrodes used for generating the current.

According to another aspect of the invention, there is provided a process for at least partially deactivating microorganisms in a solid material and dewatering the solid material, the process comprising submitting the solid material to an electric current having a voltage gradient of at least 0.5 volt per centimeter of solid material to be treated, and a current density of at least 2 mA per cm² with respect to the surface of electrodes used for generating the current density.

### DETAILED DESCRIPTION OF THE INVENTION

Further features and advantages of the invention will become more readily apparent from the following non-limiting examples.

### EXAMPLES

For Examples 1 to 4 treatments were made on a pilot scale in an activated sludge wastewater treatment plant.

The apparatus that was used in Examples 1 to 4 was an apparatus similar to the apparatus illustrated in FIGS. 13 to 18 of US 2005/0199499 and described in pages 5 to 7 of the latter document. US 2005/0199499 is hereby incorporated by reference in its entirety.

### Example 1

The process was carried out on the sludge with the previously mentioned apparatus, but with the specific parameters described in Table 1. In fact, the specific values of voltage gradient, current density, and time of treatment described in Table 1 were applied in the process and superior results in term of microorganisms deactivation were unexpectedly obtained as compared to those mentioned in US 2005/0199499. At the end of the treatment or process, a considerable increase of the sludge dryness was observed as indicated in Table 2. The electric current induced a temperature elevation. Temperatures measured in the treated sludge, at the outlet of the apparatus, varied from 70.0 to 76.3° C. Moreover, as shown in Table 3, the treatment permitted to considerably reduce the amount of microorganisms present in the sludge and event eliminate certain types of microorganisms.

TABLE 1
Treatment parameters

| Average voltage | Current density | Treatment time |
|---|---|---|
| gradient (V/cm) | (mA/cm²) | (minutes) |
| 18.7 | 6.6 | 49 |

TABLE 2
Sludge characteristics

| Properties | Before treatment | After treatment |
|---|---|---|
| pH | 7.7 | 5.7 |
| Dryness (%) | 17 | 33 |

TABLE 3
Microorganisms reduction

| | | Before | After |
|---|---|---|---|
| Microorganisms | Units | treatment | treatment |
| Escherichia coli | MPN/g dry weight | 94 × 10³ | <7 |
| Salmonella spp. | MPN/4 g dry weight | 71 | <3 |
| Enterococus | CFU/g dry weight | 447 × 10³ | <32 |
| Aerobic and facultative | CFU/g dry weight | 101 × 10⁶ | 27 × 10⁵ |
| anaerobic heterotrophic | | | |
| bacteria | | | |
| Total coliforms | MPN/g dry weight | 94 × 10³ | <7 |
| Clostridium perfringens | CFU/g dry weight | Absence | Absence |
| Somatic coliphages | PFU/4 g dry weight | 3576 | <2 |
| F-specific coliphages | PFU/4 g dry weight | 29412 | <2 |
| Enteric viruses | PFU/4 g dry weight | <0.47 | <0.24 |
| Enteric viruses | MPNIP¹/4 g dry | <0.94 | <0.24 |
| (immunoperoxydase | weight | | |
| method) | | | |
| Viable helminth ova | Ova/4 g dry weight | Absence | Absence |

¹MPNIP: Most probable number by immunoperoxidase

As it can be seen from Table 3, E. coli and Salmonella spp. levels where reduced below detection limits. For E. coli, a decrease of at least 4.13 log units was obtained, which corresponds to at least 99.99% reduction. A reduction below detection limits was obtained for: enterococus (decrease of at least 4.14 log units corresponding to at least 99.99% reduction), total coliforms (decrease of at least 4.12 log units corresponding to at least 99.99% reduction), somatic coliphages (decrease of at least 3.25 log units corresponding to at least 99.94% reduction) and F-specific coliphages (decrease of at least 4.17 log units corresponding to at least 99.99% reduction). For aerobic and facultative anaerobic heterotrophic bacteria, a decrease of 1.57 log units, corresponding to a 97.33% reduction, was obtained.

### Example 2

The sludge was treated in a similar manner as described in Example 1, but with the specific parameters described in Table 4. In fact, the specific values of voltage gradient, current density, and time of treatment described in Table 4, were applied in the process. At the end of the treatment or process, a considerable increase of the sludge dryness was observed as indicated in Table 5. The temperatures measured in the treated sludge, at the outlet of the apparatus, varied from 77.5 to 96.2° C. Table 6 shows that the treatment permitted to considerably reduce the amount of microorganisms present in the sludge and event eliminate certain types of microorganisms.

TABLE 4
Treatment parameters

| Average voltage | Maximum current | Minimum Current | Average current | Treatment time |
|---|---|---|---|---|
| gradient (V/cm) | density (mA/cm²) | density (mA/cm²) | density (mA/cm²) | (minutes) |
| 25 | 13.6 | 11 | 12.7 | 25 |

TABLE 5
Sludge characteristics

| Properties | Before treatment | After treatment |
|---|---|---|
| pH | — | 6.6 |
| Dryness (%) | 16 | 32 |

TABLE 6
Microorganisms reduction

| | | Before | After |
|---|---|---|---|
| Microorganisms | Units | treatment | treatment |
| Escherichia coli | MPN/g dry weight | 19 × 10⁵ | <7 |
| Salmonella spp. | MPN/4 g dry weight | <5 | <3 |
| Enterococus | CFU/g dry weight | 38 × 10⁴ | <32 |
| Aerobic and facultative | CFU/g dry weight | 4250 × 10³ | 4750 |
| anaerobic heterotrophic | | | |
| bacteria | | | |
| Total coliforms | MPN/g dry weight | 31 × 10⁶ | <7 |
| Clostridium perfringens | CFU/g dry weight | 775 × 10⁴ | 63 |
| Somatic coliphages | PFU/4 g dry weight | 1750 | <2.5 |
| F-specific coliphages | PFU/4 g dry weight | 6400 | <2.5 |
| Enteric viruses | PFU/4 g dry weight | <0.5 | <0.25 |
| Enteric viruses | MPNIP²/4 g dry | 1.75 | <0.25 |
| (immunoperoxydase | weight | | |
| method) | | | |
| Viable helminth ova | Ova/4 g dry weight | Absence | Absence |

During this treatment, E. coli level was reduced below detection limit. A decrease of at least 5.43 log units corresponding to at least 99.9996% was obtained. Moreover, a decrease of at least 6.65 log units was obtained for total coliforms corresponding to at least 99.9999%. For Clostridium perfringens, the results indicated a decrease of 5.09 log units, corresponding to 99.999% reduction. Enteric viruses were reduced to below the detectable limit. For aerobic and facultative anaerobic heterotrophic bacteria a decrease of 2.95 log units corresponding to 99.89% reduction was obtained by the treatment.

### Example 3

The sludge was treated in a similar manner as described in Example 1, but with the specific parameters described in Table 7. In fact, the specific values of voltage gradient, current density, and time of treatment described in Table 7, were applied in the process. At the end of the treatment or process, a considerable increase of the sludge dryness was observed as indicated in Table 8. The temperatures measured in the treated sludge, at the outlet of the apparatus, varied from 96.5 to 98.7° C. As shown in Table 9, the treatment permitted to considerably reduce the amount of microorganisms present in the sludge and event eliminate certain types of microorganisms.

TABLE 7
Treatment parameters

| Average voltage | Maximum current | Minimum current | Average current | Treatment time |
|---|---|---|---|---|
| gradient (V/cm) | density (mA/cm²) | density (mA/cm²) | density (mA/cm²) | (minutes) |
| 25.4 | 12.8 | 10.9 | 11.8 | 12.6 |

TABLE 8
Sludge characteristics

| Properties | Before treatment | After treatment |
|---|---|---|
| pH | 7.5 | 6.6 |
| Dryness (%) | 15 | 26 |

TABLE 9
Microorganisms reduction

| | | Before | After |
|---|---|---|---|
| Microorganisms | Units | treatment | treatment |
| Escherichia coli | MPN/g dry weight | 33 × 10³ | <8 |
| Salmonella spp. | MPN/4 g dry weight | 5 | <3 |
| Enterococus | CFU/g dry weight | 15 × 10⁵ | <38 |
| Aerobic and facultative | CFU/g dry weight | 547 × 10⁵ | 46 × 10² |
| anaerobic heterotrophic | | | |
| bacteria | | | |
| Total coliforms | MPN/g dry weight | >107 × 10³ | <8 |
| Clostridium perfringens | CFU/g dry weight | 550 × 10⁴ | 327 × 10² |
| Somatic coliphages | PFU/4 g dry weight | 67 × 10³ | <3 |
| F-specific coliphages | PFU/4 g dry weight | 26 × 10³ | <3 |
| Enteric viruses | PFU/4 g dry weight | 0.53 | <0.3 |
| Enteric viruses | MPNIP²/4 g dry | 5.33 | <0.3 |
| (immunoperoxydase | weight | | |
| method) | | | |
| Viable helminth ova | Ova/4 g dry weight | Absence | Absence |

In this example, enteric viruses are detected and reduced to below detection limits. For aerobic and facultative anaerobic heterotrophic bacteria, a decrease of 4.07 log units, corresponding to 99.99% reduction was obtained.

### Example 4

The sludge was treated in a similar manner as described in Example 1, but with the specific parameters described in Table 10. In fact, the specific values of voltage gradient, current density, and time of treatment described in Table 10, were applied in the process. At the end of the treatment or process, a considerable increase of the sludge dryness was observed as indicated in Table 11. As shown in Table 12, the treatment permitted to considerably reduce the fecal coliforms present in the sludge.

TABLE 10
Treatment parameters

| Average voltage | Maximum current | Minimum current | Average current | Treatment time |
|---|---|---|---|---|
| gradient (V/cm) | density (mA/cm²) | density (mA/cm²) | density (mA/cm²) | (minutes) |
| 25 | 13.4 | 10.6 | 11.6 | 38 |

TABLE 11
Sludge characteristics

| Properties | Before treatment | After treatment |
|---|---|---|
| Dryness (%) | 15 | 30 |

TABLE 12
Microorganisms reduction

| | | Before | After |
|---|---|---|---|
| Microorganisms | Units | treatment | treatment |
| Fecal coliforms | MPN/g dry | >110000 | 37 |
| | weight | | |

A reduction of fecal coliforms of at least 3.47 log units corresponding to at least 99.97% reduction was obtained by the treatment.

### Example 5

In examples 1 to 3, the analyzed samples were free of helminths ova. To show the effect of the process on helminths ova, tests were made on an activated sludge from wastewater treatment plant, which was voluntarily contaminated with helminths ova (Ascaris suis) isolated from pig faeces Wisconsin method (double centrifugation in water and after in saturated sugar solution) was used to isolate Ascaris ova (Foreyt, 2001. Veterinary Parasitology Reference Manual. 5th edition. Iowa State University Press) The solution used for sludge contamination contained approximately 1000 eggs/ml.

The apparatus used for Example 5 is an apparatus similar to the one illustrated in FIG. 2, of US 2005/0016870 and described in pages 3 and 4 of the latter document. US 2005/0016870 is hereby incorporated by reference in its entirety.

The process was carried out on the sludge with the previously mentioned apparatus but with the specific parameters described in Table 13. In fact, the specific values of voltage gradient, current density, and time of treatment described in Table 13, were applied in the process and superior results in term of microorganisms reduction were unexpectedly obtained as compared to those reported in US 2005/0016870. At the end of the treatment or process, a considerable increase of the sludge dryness was observed as indicated in Table 13. Table 13, also demonstrates that the treatment permitted to considerably reduce the amount of helminthes ova present in the sludge and even eliminate them.

TABLE 13
Results

| Treatment time | | Average voltage | Max. current | Min. current | Average current | Initial | Final | Helminths |
|---|---|---|---|---|---|---|---|---|
| (minutes) | Treatment | gradient (V/cm) | density (mA/cm²) | density (mA/cm²) | density (mA/cm²) | dryness (%) | dryness (%) | Ova/10 g |
| 13 | Treatment 1 | 24.9 | 88 | 10 | 39.4 | 14.14 | 35.31 | 0 |
| | Treatment 2 | 24.9 | 88 | 10 | 40 | 14.14 | 33.41 | 0 |
| | Treatment 3 | 24.9 | 95 | 8 | 37.8 | 14.14 | 31.78 | 0 |
| 17 | Treatment 4 | 24.9 | 95 | 10 | 35.3 | 14.14 | 39.09 | 0 |
| | Treatment 5 | 24.8 | 93 | 10 | 30.8 | 14.14 | 34.16 | 0 |
| | Treatment 6 | 24.8 | 95 | 9 | 32.9 | 14.14 | 43.79 | 0 |
| — | Untreated | — | | | — | 14.14 | — | 60 |
| | sample | | | | | | | |

Wisconsin method was used for detecting Ascaris ova in treated and untreated samples. Microscopic examination of the untreated sample shows 60 eggs of helminthes ova (Ascaris suis) by 10 grams of sludge. For treated samples (treatment 1 to 6), microscopic examination shows no eggs. These results demonstrate that such a process can permit the complete destruction or elimination of helminths ova. To check the viability of helminths ova, a culture at ambient temperature was made with eggs taken from the untreated sample. The culture showed that larval development was induced for 64% of the eggs.

### Example 6

The effluent issued from a sludge, treated with an apparatus similar to the one illustrated in FIG. 2, of US 2005/0016870 and described in pages 3 and 4 of the latter document, has been analyzed for fecal coliforms. The analysis shows less than 10 Colony Forming Unit per 100 mL. The so-obtained effluent may be used for example as a fertilizer.

TABLE 14
Treatment parameters

| Treatment time | Gradient | Maximum current | minimum current | Average current |
|---|---|---|---|---|
| (minutes) | voltage (V/cm) | density (mA/cm²) | density (mA/cm²) | density (mA/cm²) |
| 15 | 22.7 | 79 | 40 | 53 |

TABLE 15
Sludge characteristics

| Properties | Before treatment | After treatment |
|---|---|---|
| Dryness (%) | 16.37 | 38.9 |

### Example 7

Table 16 is presented in the example 7 in order to show the variation of the current densities during the treatment. Data were collected from a cell of an apparatus similar to the apparatus illustrated in FIGS. 13 to 18 of US 2005/0199499 and described in pages 5 to 7 of the latter document.

TABLE 16
Current density variation during a treatment

| | Current density |
|---|---|
| Time (minutes) | (mA/cm²) |
| 0 | 14.0 |
| 1 | 19.0 |
| 2 | 20.5 |
| 3 | 15.0 |
| 4 | 9.0 |
| 5 | 2.7 |
| 6 | 1.2 |
| 7 | 3.8 |
| 8 | 12.5 |
| 9 | 16.4 |
| 10 | 17.3 |
| 11 | 21.8 |
| 12 | 14.4 |
| 13 | 5.5 |
| Average current | 12.5 |
| density | |

### Example 8

In this example, municipal secondary sludge was treated. The apparatus that was used in Examples 8 was an apparatus similar to the apparatus illustrated in FIGS. 1 to 7 of PCT/CA2007/001052 filed on Jun. 13, 2007 and described in pages 14 to 23 of the latter document. PCT/CA2007/001052 is hereby incorporated by reference in its entirety. The total treatment process took about 15 minutes. The applied voltage gradient was about 28.6 V/cm.

During the sludge treatment, three sampling were performed. For each sampling, untreated sludge, treated sludge and generated effluent were sampled for salmonella and fecal coliforms analyses.

Tables 17 and 18 show monitoring results and various parameters obtained during the treatment of the sludge sampled at the first sampling, and for each of the five anode-units. Table 19 to 21 show microorganisms reduction for the samples taken during the first, second and third sampling.

### First Sampling

TABLE 17
Parameters used for the first, second and third anode-unit

| Anode-unit 1 | | | Anode-unit 2 | | | Anode-unit 3 | | |
|---|---|---|---|---|---|---|---|---|
| | Pres- | Current | | Pres- | Current | | Pres- | Current |
| Time | sure | density | Time | sure | density | Time | sure | density |
| (s) | (PSI) | [symbol] | (s) | (PSI) | [symbol] | (s) | (PSI) | [symbol] |
| 0 | 15.3 | 32.5 | 190 | 10.0 | 40 | 370 | 10.0 | 17.5 |
| 10 | 15.0 | 33 | 200 | 10.0 | 40 | 380 | 10.5 | 16 |
| 20 | 15.1 | 33.5 | 210 | 10.3 | 37 | 390 | 10.5 | 14.5 |
| 30 | 15.1 | 34.5 | 220 | 10.3 | 34.5 | 400 | 10.4 | 13.5 |
| 40 | 15.1 | 35 | 230 | 10.3 | 32.5 | 410 | 10.5 | 13 |
| 50 | 15.1 | 35.5 | 240 | 10.3 | 31 | 420 | 10.4 | 12.5 |
| 60 | 15.1 | 36 | 250 | 10.3 | 28.5 | 430 | 10.4 | 12.5 |
| 70 | 15.0 | 37 | 260 | 10.2 | 27 | 440 | 10.4 | 12 |
| 80 | 15.1 | 38.5 | 270 | 10.2 | 25.5 | 450 | 10.3 | 11.5 |
| 90 | 15.1 | 39 | 280 | 10.2 | 23.5 | 460 | 10.3 | 11.5 |
| 100 | 15.0 | 40 | 290 | 10.2 | 21.5 | 470 | 10.3 | 11.5 |
| 110 | 15.0 | 40 | 300 | 10.2 | 21 | 480 | 10.3 | 11 |
| 120 | 15.1 | 40 | 310 | 10.2 | 20.5 | 490 | 10.3 | 11 |
| 130 | 15.1 | 40 | 320 | 10.1 | 19.5 | 500 | 10.2 | 10.5 |
| 140 | 14.8 | 40 | 330 | 10.0 | 18.5 | 510 | 10.1 | 10.5 |
| 150 | 14.8 | 40 | 340 | 10.0 | 17.5 | 520 | 10.1 | 10.5 |
| 160 | 14.8 | 40 | 350 | 10.0 | 17.0 | 530 | 10.1 | 10.5 |
| 170 | 14.8 | 40 | 360 | 10.0 | 17.0 | 540 | 10.0 | 10.5 |
| 180 | 14.8 | 40 | — | — | — | — | — | — |

[symbol]

indicates data missing or illegible when filed

TABLE 18
Parameters used for the forth, and fifth anode-unit

| Anode-unit 4 | | | Anode-unit 5 | | |
|---|---|---|---|---|---|
| Time | Pressure | Current | Time | Pressure | Current |
| (s) | (PSI) | density (mA/cm² | (s) | (PSI) | density (mA/cm² |
| 550 | 9.2 | 26.7 | 730 | 10.6 | 12.5 |
| 560 | 9.2 | 20.0 | 740 | 10.4 | 14.0 |
| 570 | 9.3 | 13.5 | 750 | 10.3 | 12.5 |
| 580 | 9.2 | 11.5 | 760 | 10.2 | 10.5 |
| 590 | 9.9 | 10.5 | 770 | 10.2 | 9.5 |
| 600 | 9.8 | 10.0 | 780 | 13.1 | 9.0 |
| 610 | 9.7 | 9.5 | 790 | 13.0 | 9.5 |
| 620 | 9.7 | 10.0 | 800 | 13.0 | 9.0 |
| 630 | 9.5 | 9.5 | 810 | 13.0 | 8.5 |
| 640 | 9.5 | 9.0 | 820 | 13.0 | 8.0 |
| 650 | 9.5 | 9.0 | 830 | 12.9 | 8.0 |
| 660 | 9.5 | 8.5 | 840 | 12.9 | 7.5 |
| 670 | 9.3 | 8.5 | 850 | 12.8 | 7.5 |
| 680 | 9.2 | 8.5 | 860 | 12.8 | 7.2 |
| 690 | 9.1 | 8.0 | 870 | 12.7 | 7.0 |
| 700 | 9.2 | 8.5 | 880 | 12.7 | 7.0 |
| 710 | 9.7 | 8.0 | 890 | 12.6 | 7.0 |
| 720 | 9.8 | 8.5 | 900 | 12.7 | 7.0 |

TABLE 19
Microorganisms reduction

| Microorganisms | Untreated sludge | Treated sludge | Generated effluent |
|---|---|---|---|
| Fecal coliforms | >10000 MPN/g dry weight | <9 MPN/g dry weight | <10 CFU/100 ml |
| Salmonella spp. | <3 MPN/4 g dry weight | <3 MPN/4 g dry weight | <2 MPN/100 ml |

### Second Sampling

TABLE 20
Microorganisms reduction

| Microorganisms | Untreated sludge | Treated sludge | Generated effluent |
|---|---|---|---|
| Fecal coliforms | >11000 MPN/g dry weight | 10 MPN/g dry weight | <10 CFU/100 ml |
| Salmonella spp. | 293 MPN/4 g dry weight | <3 MPN/4 g dry weight | <2 MPN/100 ml |

### Third Sampling

TABLE 21
Microorganisms reduction

| Microorganisms | Untreated sludge | Treated sludge | Generated effluent |
|---|---|---|---|
| Fecal coliforms | >11000 MPN/g dry weight | <2 MPN/g dry weight | <10 CFU/100 ml |
| Salmonella spp. | 80 MPN/4 g dry weight | <3 MPN/4 g dry weight | <2 MPN/100 ml |

Examples 1 to 5, 6 and 8 thus clearly demonstrate that the processes of the present invention permit the at least partial deactivation, reduction and/or destruction of microorganisms such as E. Coli, Salmonella spp., fecal coliforms, enteric viruses and helminths ova.

The processes of the present invention can thus be useful for micoorganisms reduction or elimination in solid materials. They can also be useful for dewatering these solid materials. The solid materials can be, for example, municipal sludge, agro-alimentary sludge, industrial sludge, etc.

The processes of the present invention can also be useful for treating and/or dewatering various types of solid materials such as sediment, soil, biosolids, organic and/or inorganic sludge such as colloidal sludge, sludge from pulp and paper industries, agroalimentary sludge, sludge issued from a chemical or biological treatment, sludge from a dairy, sludge from a slaughterhouse, sludge from liquid or semi-liquid manure such as pork manure, and sludge from wastewater treatment plant. These processes can be used in industrial applications as well as for protecting environment.

While the invention has been described with particular reference to the illustrated embodiment, it will be understood that numerous modifications thereto will appear to those skilled in the art, without however departing from the scope of the claims. Accordingly, the above description and examples should be taken as illustrative of the invention and not in a limiting sense.

1. A process for at least partially deactivating microorganisms in a solid material, said process comprising submitting said solid material to an electric current having a voltage gradient of at least 3 volts per centimeter of solid material to be treated, and a current density of at least 2 mA per cm² with respect to the surface of electrodes used for generating said current.

2. A process for at least partially deactivating microorganisms in a solid material and dewatering said solid material, said process comprising submitting said solid material to an electric current having a voltage gradient of at least 3 volts per centimeter of solid material to be treated, and a current density of at least 2 mA per cm2 with respect to the surface of electrodes used for generating said current density and compacting said solid material.

3. The process of claim 1, wherein said solid material is submitted to said electrical current and compacted simultaneously.

4. The process of claim 1, wherein, said solid material is compacted by a pressure applied to it, said pressure varying according to the consistency of said solid material, the pressure increasing when the solid material consistency is increasing.

5-9. (canceled)

10. The process of claim 1, wherein the solid material is moved in a predetermined direction, and wherein the solid material is compacted by applying a pressure to the solid material in a direction which is substantially perpendicular to said predetermined direction.

11-21. (canceled)

22. The process of claim 1, wherein said voltage gradient is about 3 V/cm to about 60 V/cm.

23-32. (canceled)

33. The process of claim 1, wherein said current has an average current density of about 6 mA/cm² to about 70 mA/cm².

34-38. (canceled)

39. The process of claim 1, wherein said microorganisms comprise Escherichia coli, and wherein said process permits to reduce of at least 99.9% the content of Escherichia coli in said solid material.

40-45. (canceled)

46. The process of claim 1, wherein said microorganisms comprise salmonella, and wherein said process permits to reduce of at least 99.99% the content of salmonella in said solid material.

47-49. (canceled)

50. The process of claim 1, wherein said microorganisms comprise enterococus, and wherein said process permits to reduce of at least 99.99% the content of enterococus in said solid material.

51-53. (canceled)

54. The process of claim 1, wherein said microorganisms comprise aerobic and facultative anaerobic heterotrophic bacteria, and wherein said process permits to reduce of at least 99.99% the content of aerobic and facultative anaerobic heterotrophic bacteria in said solid material.

55-57. (canceled)

58. The process of claim 1, wherein said microorganisms comprise total coliforms, and wherein said process permits to reduce of at least 99.99% the content of total coliforms in said solid material.

59-63. (canceled)

64. The process of claim 1, wherein said microorganisms comprise fecal coliforms, and wherein said process permits to reduce of at least 99.99% the content of fecal coliforms in said solid material.

65-69. (canceled)

70. The process of claim 1, wherein said microorganisms comprise Clostrodium perfringens, and wherein said process permits to reduce of at least 99.99% the content of Clostrodium perfringens in said solid material.

71-73. (canceled)

74. The process of claim 1, wherein said microorganisms comprise somatic coliphages, and wherein said process permits to reduce of at least 99.9% the content of somatic coliphages in said solid material.

75-77. (canceled)

78. The process of claim 1, wherein said microorganisms comprise F-specific coliphages, and wherein said process permits to reduce of at least 99.99% the content of F-specific coliphages in said solid material.

79-81. (canceled)

82. The process of claim 1, wherein said microorganisms comprise enteric viruses, and wherein said process permits to reduce of at least 99.99% the content of enteric viruses in said solid material.

83-85. (canceled)

86. The process of claim 1, wherein said microorganisms comprise helminths ova, and wherein said process permits to reduce of at least 99.9% the content of helminths ova in said solid material.

87-94. (canceled)

95. The process of claim 1, wherein during said process the solid material is heated at a temperature of at least 70° C. for a period of at least 30 minutes.

96. (canceled)

97. (canceled)

98. (canceled)

99. The process of claim 1, wherein said solid material is sludge.

100-101. (canceled)
